(19) United States
(12) Patent Application Publication
(10) Pub. No.: US 2017/0247716 A1
(43) Pub. Date: Aug. 31, 2017

(54) MODULATION OF RTL GENE EXPRESSION AND IMPROVING AGRONOMIC TRAITS

(71) Applicant: PIONEER HI-BRED INTERNATIONAL, INC., Johnston, IA (US)

(72) Inventors: Rayeann Archibald, ALTOONA, IA (US); BRUCE DRUMMOND, Des Moines, IA (US); JINRUI SHI, JOHNSTON, IA (US); HONGYU WANG, JOHNSTON, IA (US)

(21) Appl. No.: 15/440,753

(22) Filed: Feb. 23, 2017

Related U.S. Application Data
(60) Provisional application No. 62/300,139, filed on Feb. 26, 2016.

(51) Int. Cl. C12N 15/82; C12Q 1/68

(52) U.S. Cl. CPC C12N 15/8261; C12N 15/8273; C12Q 1/6895; C12Q 2600/13; C12Q 2600/172

(57) ABSTRACT

Nucleotide sequences encoding RTL polypeptides are provided herein, along with plants and cells having increased levels of RTL gene expression, increased levels of RTL transcription factor activity, or both. Plants with increased levels of at least one RTL gene expression that exhibit reduced ethylene sensitivity, increased yield, increased abiotic stress tolerance, or any combination of these, are provided. Methods for increasing yield, and abiotic stress tolerance in plants, by modulating RTL gene expression or activity, are also provided.

### REFERENCE TO SEQUENCE LISTING SUBMITTED ELECTRONICALLY

The official copy of the sequence listing is submitted electronically via EFS-Web as an ASCII formatted sequence listing with a file named 5253_ST25 created on Feb. 18, 2016 and having a size of 20 kilobytes and is filed concurrently with the specification. The sequence listing contained in this ASCII formatted document is part of the specification and is herein incorporated by reference in its entirety.

The entire disclosure of the priority provisional application Ser. No. 62/300,139 filed Feb. 26, 2016 is incorporated herein by reference.

### FIELD

The field relates to plant breeding and genetics and, in particular, to recombinant DNA constructs useful in plants for modulating agronomic traits.

### BACKGROUND

Yield is a trait of particular economic interest, especially because of increasing world population and the dwindling supply of arable land available for agriculture. Crops such as corn, wheat, rice, canola and soybean account for over half the total human caloric intake, whether through direct consumption of the seeds themselves or through consumption of meat products raised on processed seeds.

Abiotic stress is the primary cause of crop loss worldwide, causing average yield losses of more than 50% for major crops (Boyer, J. S. (1982) Science 218:443-448; Bray, E. A. et al. (2000) In Biochemistry and Molecular Biology of Plants, Edited by Buchannan, B. B. et al., Amer. Soc. Plant Biol., pp. 1158-1203).

Among the various abiotic stresses, drought is a major factor that limits crop productivity worldwide. Another abiotic stress that can limit crop yields is low nitrogen stress. The adsorption of nitrogen by plants plays an important role in their growth. Plants synthesize amino acids from inorganic nitrogen in the environment. Consequently, nitrogen fertilization has been a powerful tool for increasing the yield of cultivated plants, such as maize and soybean. If the nitrogen assimilation capacity of a plant can be increased, then increases in plant growth and yield increase are also expected. Plant varieties that have tolerance to drought stress and/or better nitrogen use efficiency (NUE) are desirable.

Ethylene hormone signaling has been implicated in drought response. Modulation of ethylene sensitivity in crop plants is desirable.

### SUMMARY

The present disclosure includes:

One embodiment is a maize plant in which expression of a RTL gene is increased, when compared to a control plant, wherein the RTL gene encodes a RTL polypeptide and wherein the plant exhibits at least one phenotype selected from the group consisting of: reduced ethylene sensitivity, increased drought tolerance, increased yield, increased abiotic stress tolerance, and increased biomass compared to the control plant.

The maize plant may exhibit reduced ethylene sensitivity, increased abiotic stress tolerance, and the abiotic stress may be drought stress, low nitrogen stress, or both. The plant may exhibit the phenotype of increased yield under non-stress or stress conditions. The plant may exhibit the phenotype under drought stress conditions.

The RTL polypeptide may comprise an amino acid sequence with at least 80% sequence identity to SEQ ID NOS: 1-4.

The plant may be a monocot plant such as but not limited to a maize plant.

The increase in expression of a RTL gene may be caused by expression using a heterologous regulatory element such as for example, a promoter or an enhancer. The increase in expression of the endogenous RTL gene may also be caused by a mutation in the endogenous RTL gene or its regulatory element, and the mutation may be caused by insertional mutagenesis including but not limited to transposon mutagenesis, or it may be caused by zinc finger nuclease, Transcription Activator-Like Effector Nuclease (TALEN), CRISPR (RNA guided cas9 endonuclease) or meganuclease.

Another embodiment is a DNA construct comprising a polynucleotide, wherein the polynucleotide is operably linked to a heterologous promoter in sense orientation, wherein the construct is effective for increasing expression of a RTL gene in a plant, and wherein the polynucleotide comprises: (a) one of the nucleotide sequence of SEQ ID NOS: 5-12; (b) a nucleotide sequence that has at least 80% sequence identity, when compared to one of SEQ ID NOS: 5-12; (c) a nucleotide sequence of at least 100 contiguous nucleotides of one of SEQ ID NOS: 5-12; (d) a nucleotide sequence that can hybridize under stringent conditions with the nucleotide sequence of (a).

Another embodiment is a method of making a plant in which expression of a RTL gene is increased, when compared to a control plant, and wherein the plant exhibits at least one phenotype selected from the group consisting of: reduced ethylene sensitivity, increased yield, increased drought tolerance, increased abiotic stress tolerance, and increased biomass, compared to the control plant, the method comprising the steps of introducing into a plant a DNA construct comprising a polynucleotide operably linked to a heterologous promoter, wherein the DNA construct is effective for increasing expression of a RTL gene.

Another embodiment is a method of making a plant in which expression of an endogenous RTL gene is increased, when compared to a control plant, and wherein the plant exhibits at least one phenotype selected from the group consisting of: reduced ethylene sensitivity, increased yield, increased drought tolerance, increased abiotic stress tolerance, and increased biomass, compared to the control plant, the method comprising the steps of: (a) introducing a mutation into an endogenous RTL gene; and (b) wherein said mutation results in reducing or increasing the expression of the endogenous RTL gene.

Another embodiment is a method of enhancing seed yield in a plant, when compared to a control plant, wherein the plant exhibits enhanced yield under either drought stress conditions, or non-stress conditions, or both, the method comprising the step of increasing expression of the endogenous RTL gene in a plant.

Another embodiment is a method of making a plant in which activity of an endogenous RTL polypeptide is increased, when compared to the activity of wild-type RTL polypeptide from a control plant, and wherein the plant exhibits at least one phenotype selected from the group consisting of: reduced ethylene sensitivity, increased yield, increased drought tolerance, increased abiotic stress tolerance and increased biomass, compared to the control plant, wherein the method comprises the steps of introducing into a plant a DNA construct comprising a polynucleotide operably linked to a heterologous promoter, wherein the polynucleotide encodes a fragment or a variant of a polypeptide having an amino acid sequence of at least 80% sequence identity, when compared to SEQ ID NOS: 1-4, wherein the fragment or the variant confers a dominant-negative phenotype in the plant.

Another embodiment is a plant comprising any of the DNA constructs disclosed herein, wherein expression of the endogenous RTL gene is increased in the plant, when compared to a control plant, and wherein the plant exhibits a phenotype selected from the group consisting of: reduced ethylene sensitivity, increased yield, increased drought tolerance, increased abiotic stress tolerance and increased biomass, compared to the control plant. The plant may exhibit an increase in abiotic stress tolerance, and the abiotic stress may be drought stress, low nitrogen stress, or both or an increase in yield under normal growing conditions. The plant may exhibit the phenotype of increased yield and the phenotype may be exhibited under non-stress or stress conditions. The plant may be a monocot plant such as but not limited to a maize plant.

Another embodiment is a method of identifying one or more alleles associated with increased yield in a population of maize plants, the method comprising the steps of: (a) detecting in a population of maize plants one or more polymorphisms in (i) a genomic region encoding a polypeptide or (ii) a regulatory region controlling expression of the polypeptide, wherein the polypeptide comprises the amino acid sequence selected from the group consisting of SEQ ID NOS: 1-4, or a sequence that is 90% identical to SEQ ID NOS: 1-4, wherein the one or more polymorphisms in the genomic region encoding the polypeptide or in the regulatory region controlling expression of the polypeptide is associated with yield; and (b) identifying one or more alleles at the one or more polymorphisms that are associated with increased yield. The one or more alleles associated with increased yield may be used for marker assisted selection of a maize plant with increased yield. The one or more polymorphisms may be in the coding region of the polynucleotide. The regulatory region may be a promoter element.

Any progeny or seeds obtained from the plants disclosed herein are also provided herein.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows visualization of protein-protein interactions of maize and Arabidopsis ARGOS with AtRTE1 in Arabidopsis. (A). Fluorescence images of the lower side of leaves in Arabidopsis transgenic plants over-expressing nGFP-AtRTE1 and cGFP-tagged maize and Arabidopsis ARGOS proteins. Reconstituted green fluorescence associated with the vascular tissues is shown for representative leaves of 18-day-old F1 plants. F1 plants derived from the nGFP-AtRTE1 and cGFP-AtCb5D crosses and the AtCHX20-nGFP and ZmARGOS1-cGFP crosses serve as positive and negative controls, respectively. All images were captured with the same setting (1 s exposure time). Scale bar=500 μm. (B). Western blotting analysis of ZmARGOS1-cGFP and nGFP-AtRTE1 fusion protein expression in Arabidopsis plants. Protein extracts were prepared from leaves of a representative 18-day-old F1 plant derived from the cross of the DMMV::nGFP-AtRTE1 plants and the DMMV::ZmARGOS1-cGFP plants. Anti-HA antibodies were used to detect the fusion proteins which contain the HA epitope. (C). Visualization of Arabidopsis ARGOS homolog OSR1 and AtRTE1 interactions in Arabidopsis hypocotyl cells using the BiFC assay. Representative fluorescence images of hypocotyl cells from a 3-day-old etiolated Arabidopsis F1 seedling showing reconstituted GFP fluorescence (left) as interconnected threads and small punctate bodies within each cell. The highly vacuolate nature of these cells crowds the GFP-positive bodies toward inner perimeter of the cells. An auto-fluorescence image (middle) was captured with a near-UV (DAPI) filter set to show cell walls. At right is the merged image of GFP expression (green) and auto-fluorescence (blue). Scale bar=50 μm.

FIG. 2 shows visualization of the interaction of maize and Arabidopsis ARGOS proteins with AtRTE1 in Arabidopsis using BiFC analysis. (A). Fluorescence images of leaves of Arabidopsis transgenic plants over-expressing nGFP-AtRTE1 and cGFP-tagged maize and Arabidopsis ARGOS proteins. Reconstituted green fluorescence associated with the vascular tissues is shown for representative leaves of 18-day-old F1 plants. The F1 plants derived from the cross of the DMMV:nGFP-AtRTE1 plants and the DMMV:AtCb5D plants serve as a positive control for the BiFC assay. Scale bar=500 μm. (B). Fluorescence images of a small section near the main vein of a leaf from a 24-d-old F1 plant over-expressing nGFP-AtRTE1 and ZmARGOS8(TR)-cGFP. The lower side of the leaf was viewed under a fluorescent compound microscope. Representative fluorescence images of epidermal cells showing reconstituted GFP fluorescence (left) captured using a GFP filter (Chroma Technology, #41001; Excitation=460-500 nm; Dichroic=505LP; Emission=510-560 nm). An auto-fluorescence image (middle) was captured using a DAPI bandpass filter (Chroma Technology, #31013; Excitation=360-370 nm; Dichroic=380LP; Emission=435-485 nm). At right is the merged image of GFP expression (green) and auto-fluorescence (blue). Scale bar=50 μm.

FIG. 3 shows interactions of Arabidopsis and maize ARGOS with AtRTE1 as revealed with the yeast split ubiquitin system. (A) Growth of yeast diploid cells on selective synthetic complete (SC) dropout media to show protein-protein interactions. Diploid cells were generated by mating haploid strain THY.AP4 containing the bait construct of AtRTE1-Cub-PLV with THY.AP5 carrying the prey constructs of Arabidopsis or maize ARGOS and NubG fusions. Empty vector NubG serves as a control for autoactivation of the bait AtRTE1-Cub-PLV. The prey construct AtARGOS-NubWT is included to show that the AtRTE1-Cub-PLV fusion protein is properly expressed and cleaved PLV is functional (also see C). Serial dilutions of liquid cultures were spotted on the indicated plates and incubated at 28° C. for 4 days. Upper panel shows growth and accumulation of red pigments in yeast cells on adenine and histidine-supplemented SC dropout medium. Lower panel shows growth on histidine selective medium reporting interactions of AtRTE1 with various ARGOS proteins. (B) β-Galactosidase assay for yeast diploid cells expressing AtRTE1-Cub-PLV and various ARGOS-NubG fusion proteins. Yeast cells were cultured in SC-Leu-Trp liquid medium. The β-Galactosidase (β-Gal) activity assay uses o-nitrophenylglucoside as a substrate. (C) Growth of yeast diploid cells from the mating of haploid strains containing ARGOS-NubG constructs and negative control AtCHX20-Cub-LPV construct. The plates were incubated at 28° C. for 4 days. (D) Liquid β-Galactosidase assay for yeast diploid cells expressing negative control AtCHX20-Cub-PLV and various ARGOS-NubG fusion proteins.

FIG. 4 shows interactions of Arabidopsis and maize ARGOS with ZmRTL4 and ZmRTL2 in the yeast split-ubiquitin assay. (A) Growth of yeast diploid cells on SC dropout media to show protein-protein interaction of ZmRTL4 with Arabidopsis and maize ARGOS. The SC-Leu-Trp and SC-Leu-Trp-His-Ade media contain 134 and 375 μM Met, respectively. The plates were incubated at 28° C. for 4 days. (B) β-Galactosidase assay for yeast diploid cells expressing ZmRTE4-Cub-PLV and various ARGOS-NubG fusion proteins. (C) Growth of yeast diploid cells on SC dropout media to show protein-protein interaction of ZmRTL2 with Arabidopsis and maize ARGOS. The SC-Leu-Trp and SC-Leu-Trp-His-Ade media contain 134 μM Met. The plates were incubated at 28° C. for 5 days. (D) β-Galactosidase assay for yeast diploid cells expressing ZmRTE2-Cub-PLV and various ARGOS-NubG fusion proteins.

FIG. 5 shows visualization of the interaction of maize ARGOS and RTL proteins in Arabidopsis using the BiFC assay. Representative fluorescence images are shown for leaves of the 18-day-old F1 Arabidopsis transgenic plants over-expressing nGFP-tagged ZmRTL4 and ZmRTL2 and cGFP-tagged ZmARGOS1, ZmARGOS8 and ZmARGOS8(TR).

FIG. 6 shows overexpression of maize RTL2 and RTL4 reduces ethylene sensitivity in maize plants. (A) Root lengths of etiolated seedlings of transgenic maize. The UBI1:ZmRTL2 and UBI1:ZmRTL4 plants were grown in a filter-paper roll set vertically in the dark in the presence of 0 or 100 μM ACC for 5 days. The primary roots of 15 seedlings per event per treatment were measured. Data show means+SD of five events. Student's t test was performed to compare the transgenic plants with nulls. *, P<=0.05. (B) Ethylene emission from leaves of UBI1:ZmRTL2 and UBI1:ZmRTL4 transgenic events and non-transgenic (Null) control. Leaf discs were taken from the seventh leaf of V8 plants grown in greenhouse. Ethylene was collected for a period of 22-24 h and subsequently measured using a gas chromatograph. Error bars indicate SD; n=35 for nulls and 9 for each transgenic event. Comparison of the transgenic plants with nulls found no significant difference (Student's t test,P>0.05).

FIG. 7 illustrates Sequence analysis of Arabidopsis and maize RTE1 proteins. (A) A guide tree displaying the degree of similarity among protein sequences. The calculated distance values are shown in parenthesis. (B). Percentage of identical aminol acid residues in the alignment.

FIG. 8 shows maize RTL gene expression in B73 inbred. Transcript abundance was measured with RNA sequencing. RPKtM, reads per kilobase of transcript per ten million mapped reads.

FIG. 9 shows phylogenetic relationship of maize RTL proteins and other RTE1 homologs. A neighbor-joining phylogenetic tree based on a ClustalW multiple sequence alignment was constructed using MEGA version 6 (Tamura et al. 2013, Molecular Biology and Evolution: 30 2725-2729). Thirty-one plant RTE1 proteins were used: AtRTE1 (AEC07792) and AtRTH (AY045821) from Arabidopsis; ZmRTL1 (NM_001151994), ZmRTL2 (BT036282), ZmRTL3 (ACN37097) and ZmRTL4 (NM_001150599) from maize; SiRTL1 (XP_004961368) and SiRTL2 (XP_004981412) from Setaria italica; SbRTL1 (EES01361), SbRTL2 (Sobic.009G213600) and SbRTL3 (EER93278) from sorghum; ObRTL2 (XP_006650739) from Oryza brachyantha; OsRTH1 (BAB39426), OsRTH2 (AAV59409) and OsRTH3 (AAO37528) from rice; HvRTL1 (BAK01520) from barley; BdRTL1 (XP_003569673) and BdRTL2 (XP_003557262) from Brachypodium distachyon; MdRTL1 (XP_008393649) and MdRTL2 (XP_008337804) from Malus domestica; GmRTL1 (ACU21237), GmRTL2 (ACU18547) and GmRTL3 (ACU21338) from soybean; PvRTL1 (ESW32996) and PvRTL2 (ESW30603) from Phaseolus vulgaris; SIGR (ABD34614), SIGRL1 (ABD34616) and SIGRL2 (ABD34617) from tomato; PhGR (AFX95946), PhGRL1 (AFX95947) and PhGRL2 (AIA08937) from petunia.

Table 1 presents SEQ ID NOs for the CDS sequences of other RTL family members from Zea mays. The SEQ ID NOs for the corresponding amino acid sequences encoded by the cDNAs are also presented.

TABLE 1
CDS sequences Encoding maize RTL Polypeptides

| Plant | Clone Designation | SEQ ID NO: |
|---|---|---|
| Maize | ZmRTL4 polypeptide | 1 |
| Maize | ZmRTL1 polypeptide | 2 |
| Maize | ZmRTL2 polypeptide | 3 |
| Maize | ZmRTL3 polypeptide | 4 |
| Maize | ZmRTL4 CDS | 5 |
| Maize | ZmRTL1 CDS | 6 |
| Maize | ZmRTL2 CDS | 7 |
| Maize | ZmRTL3 CDS | 8 |
| Maize | ZmRTL4 cDNA | 9 |
| | (includes UTRs) | |
| Maize | ZmRTL1 cDNA | 10 |
| | (includes UTRs) | |
| Maize | ZmRTL2 cDNA | 11 |
| | (includes UTRs) | |
| Maize | ZmRTL3 cDNA | 12 |
| | (includes UTRs) | |

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

The phytohormone ethylene regulates plant growth and development as well as plant response to environmental cues. The sensitivity of plants to ethylene is reduced in transgenic Arabidopsis overexpressing ARGOS genes from Arabidopsis and maize. ZmARGOS1, as well as Arabidopsis ARGOS homolog ORGAN SIZE RELATED1 (OSR1), physically interacts with REVERSION-TO-ETHYLENE SENSITIVITY1 (RTE1), an ethylene receptor interacting protein which regulates the activity of ETHYLENE RESPONSE1 (ETR1). The protein-protein interaction was confirmed with the yeast split-ubiquitin two-hybrid system. Maize REVERSION-TO-ETHYLENE SENSITIVITY1 LIKE4 (ZmRTL4), an AtRTE1 homolog, also interacts with ZmARGOS1, ZmARGOS8 and Arabidopsis ARGOS proteins. Like AtRTE1 in Arabidopsis, ZmRTL4 reduces ethylene sensitivity when overexpressed in maize, indicating a similar mechanism for ARGOS regulating ethylene signaling in maize. A polypeptide fragment derived from ZmARGOS8, comprising a proline-rich motif flanked by two transmembrane helices which are conserved among members of the ARGOS family, can interact with AtRTE1 and ZmRTL4 in the Arabidopsis and yeast assays. The conserved domain is necessary and sufficient to confer ethylene insensitivity in Arabidopsis and maize. Overall, these results suggest a physical association between ARGOS and the ethylene receptor signaling complex via RTE1, supporting a role for ARGOS in regulating ethylene perception and the early steps of signal transduction in Arabidopsis and maize.

As used herein:

The term “RTL gene” refers herein to the gene that encodes for one or more of the RTL polypeptides disclosed herein. In an embodiment, the term “RTL polypeptide” refers herein to a polypeptide that is homologous to Arabidopsis REVERSION-TO-ETHYLENE SENSITIVITY1 (RTE1), an ethylene receptor interacting protein which regulates the activity of ETHYLENE RESPONSE1 (ETR1). Maize REVERSION-TO-ETHYLENE SENSITIVITY1 LIKE4 (ZmRTL4) is an AtRTE1 homolog is represented by SEQ ID NO: 1 and. The term “RTL polypeptide”, as referred to herein is a polypeptide comprising an amino acid sequence with at least 80% sequence identity to SEQ ID NOS: 1-4.

The terms “monocot” and “monocotyledonous plant” are used interchangeably herein. A monocot of the current disclosure includes the Gramineae or Poaceae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” generally refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS). Nutrients include, but are not limited to, the following: nitrogen (N), phosphorus (P), potassium (K), calcium (Ca), magnesium (Mg) and sulfur (S). For example, the abiotic stress may be drought stress, low nitrogen stress, or both.

“Nitrogen limiting conditions” or “low nitrogen stress” refers to conditions where the amount of total available nitrogen (e.g., from nitrates, ammonia, or other known sources of nitrogen) is not sufficient to sustain optimal plant growth and development. One skilled in the art would recognize conditions where total available nitrogen is sufficient to sustain optimal plant growth and development. One skilled in the art would recognize what constitutes sufficient amounts of total available nitrogen, and what constitutes soils, media and fertilizer inputs for providing nitrogen to plants. Nitrogen limiting conditions will vary depending upon a number of factors, including but not limited to, the particular plant and environmental conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

“Nitrogen stress tolerance” is a trait of a plant and refers to the ability of the plant to survive under nitrogen limiting conditions over prolonged periods of time, without exhibiting the same degree of physiological or physical deterioration relative to the reference or control plant grown under similar stress conditions.

“Increased nitrogen stress tolerance” of a plant is measured relative to a reference or control plant, and means that the nitrogen stress tolerance of the plant is increased by any amount or measure when compared to the nitrogen stress tolerance of the reference or control plant.

A “nitrogen stress tolerant plant” is a plant that exhibits nitrogen stress tolerance. A nitrogen stress tolerant plant may be a plant that exhibits an increase in at least one agronomic characteristic relative to a control plant under nitrogen limiting conditions.

“Environmental conditions” refer to conditions under which the plant is grown, such as the availability of water, availability of nutrients (for example nitrogen), or the presence of insects or disease.

“Stay-green” or “staygreen” is a term used to describe a plant phenotype, e.g., whereby leaf senescence (most easily distinguished by yellowing of leaf associated with chlorophyll degradation) is delayed compared to a standard reference or a control. The staygreen phenotype has been used as selective criterion for the development of improved varieties of crop plants such as corn, rice and sorghum, particularly with regard to the development of stress tolerance, and yield enhancement.

“Increase in staygreen phenotype” as referred to in here, indicates retention of green leaves, delayed foliar senescence and significantly healthier canopy in a plant, compared to control plant.

The growth and emergence of maize silks play a role in the determination of yield under drought. When soil water deficit occurs before flowering, silk emergence out of the husks may be delayed while anthesis is largely unaffected, resulting in an increased anthesis-silking interval (ASI). Selection for reduced ASI has been used successfully to increase drought tolerance of maize.

Terms used herein to describe thermal time include “growing degree days” (GDD), “growing degree units” (GDU) and “heat units” (HU).

“Transgenic” generally refers to any cell, cell line, callus, tissue, plant part or plant, the genome of which has been altered by the presence of a heterologous nucleic acid, such as a DNA construct or a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a DNA construct or a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. In case of DNA constructs, as disclosed herein, gene stacking approach may encompass expression of more than one RTL gene, or may also refer to stacking of a DNA construct with a recombinant DNA construct that leads to overexpression of a particular gene or polypeptide. Gene stacking can be accomplished by many means including but not limited to co-transformation, retransformation, and crossing lines with different transgenes or breeding with other drought tolerance varieties displaying non-transgenic traits, such as for example, native drought tolerance.

The DNA constructs and nucleic acid sequences of the current disclosure may be used in combination (“stacked”) with other polynucleotide sequences of interest in order to create plants with a desired phenotype. The desired combination may affect one or more traits; that is, certain combinations may be created for modulation of gene expression affecting RTL gene activity or expression. Other combinations may be designed to produce plants with a variety of desired traits including but not limited to increased yield and altered agronomic characteristics. “Transgenic plant” also includes reference to plants which comprise more than one heterologous polynucleotide within their genome. Each heterologous polynucleotide may confer a different trait to the transgenic plant.

In an embodiment, a DNA construct comprising a polynucleotide, wherein the polynucleotide encodes a polypeptide comprising a sequence that is at least 95% identical to SEQ ID NO: 1, is operably linked in sense orientation to a heterologous promoter, wherein the expression of the polynucleotide in a maize results in an increased yield of at least about 5% as compared to a control plant not expressing the polypeptide. In an embodiment, the yield gain is about 1%, 2%, 3%, 4%, 5%, 6%, 7%, 8%, 9% or 10% as compared to the control plant not expressing the RTL gene to a level expressed by the plant in consideration.

The term “endogenous” relates to any gene or nucleic acid sequence that is already present in a cell.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” generally refers to the RNA that is without introns and that can be translated into protein by the cell.

“cDNA” generally refers to a DNA that is complementary to and synthesized from a mRNA template using the enzyme reverse transcriptase. The cDNA can be single-stranded or converted into the double-stranded form using the Klenow fragment of DNA polymerase I.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein generally refers to a post-translationally processed polypeptide; i.e., one from which any pre- or pro-peptides present in the primary translation product have been removed.

“Precursor” protein generally refers to the primary product of translation of mRNA; i.e., with pre- and pro-peptides still present. Pre- and pro-peptides may be and are not limited to intracellular localization signals.

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

As used herein the terms non-genomic nucleic acid sequence or non-genomic nucleic acid molecule generally refer to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with a genomic nucleic acid sequence; insertion of one or more heterologous introns; deletion of one or more upstream or downstream regulatory regions associated with a genomic nucleic acid sequence; insertion of one or more heterologous upstream or downstream regulatory regions; deletion of the 5′ and/or 3′ untranslated region associated with a genomic nucleic acid sequence; and insertion of a heterologous 5′ and/or 3′ untranslated region.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

“DNA construct” is a recombinant DNA construct which when transformed or stably integrated into the genome of the plant, results in the desired expression or silencing of a gene in the plant.

The terms “reference”, “reference plant”, “control”, “control plant”, “wild-type” or “wild-type plant” are used interchangeably herein, and refers to a parent, null, or non-transgenic plant of the same species that lacks the expression of the corresponding RTL gene. A control plant as defined herein is a plant that is not made according to any of the methods disclosed herein. A control plant can also be a parent plant that contains a wild-type allele of a RTL gene. A wild-type plant would be: (1) a plant that carries the unaltered or not modulated form of a gene or allele, or (2) the starting material/plant from which the plants produced by the methods described herein are derived.

Various assays for measuring gene expression are well known in the art and can be done at the protein level (examples include, but are not limited to, Western blot, ELISA) or at the mRNA level such as by RT-PCR.

In certain aspects of the disclosure, the DNA construct is sense or antisense DNA construct.

A polynucleotide sequence is said to “encode” a sense or antisense RNA molecule, or RNA silencing or interference molecule or a polypeptide, if the polynucleotide sequence can be transcribed (in spliced or unspliced form) and/or translated into the RNA or polypeptide, or a subsequence thereof.

“Expression of a gene” or “expression of a nucleic acid” means transcription of DNA into RNA (optionally including modification of the RNA, e.g., splicing), translation of RNA into a polypeptide (possibly including subsequent modification of the polypeptide, e.g., posttranslational modification), or both transcription and translation, as might be indicated by the context.

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes. Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

Gene Disruption Techniques:

The expression or activity of the RTL gene and/or polypeptide can be modulated by modifying the gene encoding the RTL polypeptide or a regulatory element of the endogenous gene. One way of modulating a gene expression is by insertional mutagenesis. The gene can be modulated by mutagenizing the plant or plant cell using random or targeted mutagenesis.

“TILLING” or “Targeting Induced Local Lesions IN Genomics” refers to a mutagenesis technology useful to generate and/or identify, and to eventually isolate mutagenized variants of a particular nucleic acid with modulated expression and/or activity (McCallum et al., (2000), Plant Physiology 123:439-442; McCallum et al., (2000) Nature Biotechnology 18:455-457; and, Colbert et al., (2001) Plant Physiology 126:480-484).

The plant containing the mutated RTL gene can be crossed with other plants to introduce the mutation into another plant. This can be done using standard breeding techniques.

Homologous recombination allows introduction in a genome of a selected nucleic acid at a defined selected position. Homologous recombination has been demonstrated in plants. See, e.g., Puchta et al. (1994), Experientia 50: 277-284; Swoboda et al. (1994), EMBO J. 13: 484-489; Offringa et al. (1993), Proc. Natl. Acad. Sci. USA 90: 7346-7350; Kempin et al. (1997) Nature 389:802-803; and, Terada et al., (2002) Nature Biotechnology, 20(10):1030-1034).

Methods for performing homologous recombination in plants have been described not only for model plants (Offringa et al. (1990) EMBO J. October; 9(10):3077-84) but also for crop plants, for example rice (Terada R, Urawa H, Inagaki Y, Tsugane K, Iida S. Nat Biotechnol. 2002 20(10):1030-4; Iida and Terada: Curr Opin Biotechnol. 2004 April; 15(2):1328). The nucleic acid to be introduced (which may be RTL nucleic acid or a variant thereof) need not be targeted to the locus of the RTL gene, but may be introduced into, for example, regions of high expression. The nucleic acid to be introduced may be a dominant negative allele used to replace the endogenous gene or may be introduced in addition to the endogenous gene.

Another way of introducing gene disruptions into a RTL gene can be by introducing site-specific mutations into RTL genes. Mutations can be introduced in the RTL gene by using proteins that can introduce DNA damage into preselected regions of the plant genome. Such proteins or catalytic domains are sometimes referred to as “DNA mutator enzymes”. The DNA damage can lead to a DSB (double strand break) in double stranded DNA). The DNA mutator enzyme domain may be fused to a protein that binds to specific DNA sites.

Examples of DNA mutator enzyme domains include, but are not limited to catalytic domains such as DNA glycolases, DNA recombinase, transposase, and DNA nucleases (PCT publication No. WO2014127287; U.S. Patent Publication No. U.S.20140087426; incorporated herein by reference).

DNA nuclease domains are another type of enzymes that can be used to introduce DNA damage or mutation. A DNA nuclease domain is an enzymatically active protein or fragment thereof that causes DNA cleavage resulting in a DSB.

DNA nucleases and other mutation enzyme domains may be fused with DNA binding domains to produce the DSBs in the target DNA. DNA binding domains include, for example, an array specific DNA binding domain or a site-specific DNA binding domain. Site specific DNA binding domain include but are not limited to a TAL (Transcription Activator-Like Effector) or a zinc finger binding domain.

Examples of DNA-binding domains fused to DNA nucleases include but are not limited to TALEN and multiple TALENs. Transcription Activator-Like Effector Nucleases (TALENs) are artificial restriction enzymes generated by fusing the TAL effector DNA binding domain to a DNA enzyme domain. TAL proteins are produced by bacteria and include a highly conserved 33-34 amino acid DNA binding domain sequence (PCT publication No. WO2014127287; U.S. Patent Publication No. U.S.20140087426).

The original TALEN chimera were prepared using the wild-type Fokl endonuclease domain. However, TALEN may also include chimera made from Fok1 endonuclease domain variants with mutations designed to improve cleavage specificity and cleavage activity. In some instances multiple TALENs can be expressed to target multiple genomic regions.

A zinc finger is another type of DNA binding domain that can be used for introducing mutations into the target DNA.

Various protein engineering techniques can be used to alter the DNA-binding specificity of zinc fingers and tandem repeats of such engineered zinc fingers can be used to target desired genomic DNA sequences. Fusing a second protein domain such as a transcriptional repressor to a zinc finger that can bind near the promoter of the YEP gene can reduce the expression levels of RTL gene.

The proteins of the CRISPR (clustered regularly interspaced short palindromic repeat) system are examples of other DNA-binding and DNA-nuclease domains. The expression levels of RTL gene or the activity of the RTL polypeptide can be increased by introducing mutations through CRISPR (clustered regularly interspaced short palindromic repeat)/Cas9 system. The bacterial CRISPR/Cas system involves the targeting of DNA with a short, complementary single stranded RNA (CRISPR RNA or crRNA) that localizes the Cas9 nuclease to the target DNA sequence (Burgess DJ (2013) Nat Rev Genet 14:80; PCT publication No. WO2014/127287). The crRNA can bind on either strand of DNA and the Cas9 will cleave the DNA making a DSB.

The present disclosure encompasses variants and subsequences of the polynucleotides and polypeptides described herein.

The term “variant” with respect to a polynucleotide or DNA refers to a polynucleotide that contains changes in which one or more nucleotides of the original sequence is deleted, added, and/or substituted while substantially maintaining the function of the polynucleotide. For example, a variant of a promoter that is disclosed herein can have minor changes in its sequence without substantial alteration to its regulatory function.

The term “variant” with respect to a polypeptide refers to an amino acid sequence that is altered by one or more amino acids with respect to a reference sequence. The variant can have “conservative changes, wherein a substituted amino acid has similar structural or chemical properties, for example, and replacement of leucine with isoleucine. Alternatively, a variant can have “non-conservative” changes, for example, replacement of a glycine with a tryptophan. Analogous minor variation can also include amino acid deletion or insertion, or both.

Guidance in determining which nucleotides or amino acids for generating polynucleotide or polypeptide variants can be found using computer programs well known in the art.

The terms “fragment” and “subsequence” are used interchangeably herein, and refer to any portion of an entire sequence.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” generally refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” generally refers to a promoter whose activity is determined by developmental events.

“Operably linked” generally refers to the association of nucleic acid fragments in a single fragment so that the function of one is regulated by the other. For example, a promoter is operably linked with a nucleic acid fragment when it is capable of regulating the transcription of that nucleic acid fragment.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” generally refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

Allelic variants encompass Single nucleotide polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

Plant breeding techniques known in the art and used in the maize plant breeding program include, but are not limited to, recurrent selection, bulk selection, mass selection, backcrossing, pedigree breeding, open pollination breeding, restriction fragment length polymorphism enhanced selection, genetic marker enhanced selection, double haploids and transformation. Often combinations of these techniques are used.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Turning now to the embodiments:

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

In one embodiment, a plant in which expression of a RTL gene is increased, when compared to a control plant, wherein the RTL gene encodes a RTL polypeptide and wherein the plant exhibits at least one phenotype selected from the group consisting of: increased yield, increased abiotic stress tolerance, and increased biomass compared to the control plant.

In one embodiment, a plant in which activity of a RTL polypeptide is increased, when compared to the activity of wild-type RTL polypeptide in a control plant, wherein the plant exhibits at least one phenotype selected from the group consisting of: increased yield, increased abiotic stress tolerance, and increased biomass compared to the control plant.

In one embodiment, the plant exhibits increased abiotic stress tolerance, and the abiotic stress is drought stress, low nitrogen stress, or both. In one embodiment, the plant exhibits the phenotype of increased yield and the phenotype is exhibited under non-stress conditions. In one embodiment, the plant exhibits the phenotype of increased yield and the phenotype is exhibited under stress conditions. In one embodiment, the plant exhibits the phenotype under drought stress conditions.

In one embodiment, the plant is a monocot plant. In another embodiment, the plant is a maize plant.

In one embodiment, the mutation in the endogenous RTL gene is caused by zinc finger nuclease, Transcription Activator-Like Effector Nuclease (TALEN), CRISPR or meganuclease.

Isolated Polynucleotides and Polypeptides:

The present disclosure includes the following isolated polynucleotides and polypeptides:

An isolated polynucleotide comprising: (i) a nucleic acid sequence encoding a RTL polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NOS: 1-11, and combinations thereof; or (ii) a full complement of the nucleic acid sequence of (i), wherein the full complement and the nucleic acid sequence of (i) consist of the same number of nucleotides and are 100% complementary. Any of the foregoing isolated polynucleotides or a fragment or subsequence of the isolated polynucleotides may be utilized in any DNA constructs of the present disclosure.

An isolated polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NOS: 1-4, and combinations thereof. The polypeptide is preferably a RTL polypeptide.

An isolated polynucleotide comprising (i) a nucleic acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NOS: 5-12, and combinations thereof; (ii) a full complement of the nucleic acid sequence of (i); or (iii) a fragment or subsequence of the nucleic acid sequence of (i). Any of the foregoing isolated polynucleotides or a fragment of the isolated polynucleotides may be utilized in any DNA construct of the present disclosure. The isolated polynucleotide preferably encodes a RTL polypeptide.

An isolated polynucleotide comprising a nucleotide sequence, wherein the nucleotide sequence is hybridizable under stringent conditions with a DNA molecule comprising the full complement of one of SEQ ID NOS: 5-12, or a subsequence thereof. The isolated polynucleotide preferably encodes a RTL polypeptide.

An isolated polynucleotide comprising a nucleotide sequence, wherein the nucleotide sequence is derived from one of SEQ ID NOS: 5-12 by alteration of one or more nucleotides by at least one method selected from the group consisting of: deletion, substitution, addition and insertion. The isolated polynucleotide preferably encodes a RTL polypeptide. An isolated polynucleotide comprising a nucleotide sequence, wherein the nucleotide sequence corresponds to an allele of one of SEQ ID NOS: 5-12.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current disclosure may also be a protein which comprises an amino acid sequence comprising deletion, substitution, insertion and/or addition of one or more amino acids in an amino acid sequence presented in SEQ ID NOS: 1-4. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present disclosure may also be a protein which is encoded by a nucleic acid comprising a nucleotide sequence comprising deletion, substitution, insertion and/or addition of one or more nucleotides in the nucleotide sequence of one of SEQ ID NOS: 5-12. Nucleotide deletion, substitution, insertion and/or addition may be accomplished by site-directed mutagenesis or other techniques as mentioned above.

The protein of the present disclosure may also be a protein which is encoded by a nucleic acid comprising a nucleotide sequence hybridizable under stringent conditions with the complementary strand of the nucleotide sequence of one of SEQ ID NOS: 5-12.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Recombinant DNA Constructs and DNA Constructs:

In one aspect, the present disclosure includes DNA constructs.

One embodiment is a DNA construct comprising a polynucleotide, wherein the polynucleotide is operably linked to a heterologous promoter in sense or antisense orientation, or both, wherein the construct is effective for reducing expression of an endogenous RTL gene in a plant, and wherein the polynucleotide comprises: (a) the nucleotide sequence of one of SEQ ID NOS: 5-12; (b) a nucleotide sequence that has at least 80% sequence identity, when compared to one of SEQ ID NOS: 5-12; (c) a nucleotide sequence of at least 100 contiguous nucleotides of one of SEQ ID NOS: 5-12; (d) a nucleotide sequence that can hybridize under stringent conditions with the nucleotide sequence of (a); or (e) a modified plant miRNA precursor, wherein the precursor has been modified to replace the miRNA encoding region with a sequence designed to produce a miRNA directed to one of SEQ ID NOS: 5-12.

In one embodiment, the RTL polypeptide may be from Zea mays, Glycine max, Oryza sativa, Sorghum bicolor, Saccharum officinarum, or Triticum aestivum.

In one embodiment, the promoter may be a constitutive promoter, an inducible promoter, a tissue-specific promoter.

Regulatory Sequences:

A recombinant DNA construct (including a DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance stress tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)). Endosperm preferred promoters include those described in e.g., U.S. Pat. No. 8,466,342; U.S. Pat. No. 7,897,841; and U.S. Pat. No. 7,847,160.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Promoters for use also include the following: Zm-GOS2 (maize promoter for “Gene from Oryza sativa” (see e.g., U.S. Pat. No. 6,504,083 B1), U.S. publication number U.S.2012/0110700 for Sb-RCC (Sorghum promoter for Root Cortical Cell delineating protein, root specific expression), Zm-ADF4 (U.S. Pat. No. 7,902,428; Maize promoter for Actin Depolymerizing Factor), Zm-FTM1 (U.S. Pat. No. 7,842,851; maize promoter for Floral transition MADSs) promoters; OsActin promoter (WO2014160304—SEQ ID NO: 4).

Additional promoters for regulating the expression of the nucleotide sequences in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one enhancer element; e.g., a 35S, nos or ocs enhancer element.

Promoters for use may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (U.S. 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

The promoters disclosed herein may be used with their own introns, or with any heterologous introns to drive expression of the transgene.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10: 107-117).

Examples of terminators for use include, but are not limited to, PinII terminator, SB-GKAF terminator (U.S. Appln. No. 61/514,055), Actin terminator, Os-Actin terminator, Ubi terminator, Sb-Ubi terminator, Os-Ubi terminator.

A composition of the present disclosure is a plant comprising in its genome any of the DNA constructs of the present disclosure (such as any of the constructs discussed above). Compositions also include any progeny of the plant, and any seed obtained from the plant or its progeny, wherein the progeny or seed comprises within its genome the DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced DNA construct. These seeds can be grown to produce plants that would exhibit an altered agronomic characteristic (e.g., an increased agronomic characteristic optionally under stress conditions), or used in a breeding program to produce hybrid seed, which can be grown to produce plants that would exhibit such an altered agronomic characteristic. The seeds may be maize seeds. The stress condition may be selected from the group of drought stress, and nitrogen stress.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

In any of the embodiments described herein, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

In any of the embodiments described herein, the plant may exhibit less yield loss relative to the control plants, for example, at least 25%, at least 20%, at least 15%, at least 10% or at least 5% less yield loss, under stress conditions. The stress may be either drought stress, low nitrogen stress, or both.

In one embodiment, the plant may exhibit increased yield, for example, at least 5%, at least 10%, at least 15%, at least 20% or at least 25% increased yield, relative to the control plants under non-stress conditions.

Yield analysis can be done to determine whether plants that have expression levels of at least one of the RTL genes have an improvement in yield performance under non-stress or stress conditions, when compared to the control plants that have wild-type expression levels and activity levels of the YEP gene and polypeptide, respectively. Stress conditions can be water-limiting conditions, or low nitrogen conditions. Specifically, drought conditions or nitrogen limiting conditions can be imposed during the flowering and/or grain fill period for plants that contain the DNA construct and the control plants.

In one embodiment, the plant may exhibit phenotype, or an increase in biomass, relative to the control plants under non-stress conditions.

In one embodiment, the plant may exhibit phenotype, or an increase in biomass, relative to the control plants under stress conditions.

In one embodiment, yield can be measured in many ways, including, for example, test weight, seed weight, seed number per plant, seed number per unit area (i.e. seeds, or weight of seeds, per acre), bushels per acre, tonnes per acre, tons per acre, kilo per hectare.

The terms “stress tolerance” or “stress resistance” as used herein generally refers to a measure of a plants ability to grow under stress conditions that would detrimentally affect the growth, vigor, yield, and size, of a “non-tolerant” plant of the same species. Stress tolerant plants grow better under conditions of stress than non-stress tolerant plants of the same species. For example, a plant with increased growth rate, compared to a plant of the same species and/or variety, when subjected to stress conditions that detrimentally affect the growth of another plant of the same species would be said to be stress tolerant. A plant with “increased stress tolerance” can exhibit increased tolerance to one or more different stress conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions. Typically, when a transgenic plant comprising a recombinant DNA construct or DNA construct in its genome exhibits increased stress tolerance relative to a reference or control plant, the reference or control plant does not comprise in its genome the recombinant DNA construct or DNA construct.

“Drought” generally refers to a decrease in water availability to a plant that, especially when prolonged, can cause damage to the plant or prevent its successful growth (e.g., limiting plant growth or seed yield). “Water limiting conditions” generally refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or DNA construct in its genome exhibits increased drought tolerance relative to a reference or control plant, the reference or control plant does not comprise in its genome the recombinant DNA construct or DNA construct.

When a transgenic plant comprising a DNA construct in its genome exhibits increased stress tolerance relative to a reference or control plant, the reference or control plant does not comprise in its genome the DNA construct.

The range of stress and stress response depends on the different plants which are used, i.e., it varies for example between a plant such as wheat and a plant such as Arabidopsis.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Transformation of Maize Using Agrobacterium

Maize plants can be transformed with the DNA construct containing ZmRTL or a DNA construct containing any of the corresponding homologs from maize (from Table 1) in order to examine the resulting phenotype.

Agrobacterium-mediated transformation of maize is performed essentially as described by Zhao et al. in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium inoculation, co-cultivation, resting, selection and plant regeneration.

1. Immature Embryo Preparation:

Immature maize embryos are dissected from caryopses and placed in a 2 mL microtube containing 2 mL PHI-A medium.

2. Agrobacterium Infection and Co-Cultivation of Immature Embryos:

2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

2.2 Co-culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

Media for Plant Transformation:
1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L GELRITE®, 100 μM acetosyringone (filter-sterilized), pH 5.8.
3. PHI-C: PHI-B without GELRITE® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a DNA construct can be introduced into an elite maize inbred line either by direct transformation or introgression from a separately transformed line.

Transgenic plants, either inbred or hybrid, can undergo more vigorous field-based experiments to study yield enhancement and/or stability under water limiting and water non-limiting conditions.

Subsequent yield analysis can be done to determine whether plants that contain the increased expression levels or increased activity of RTL genes have an improvement in yield performance (under stress or non-stress conditions), when compared to the control (or reference) plants that do not contain the DNA construct. Specifically, water limiting conditions can be imposed during the flowering and/or grain fill period for plants that have increased expression or activity levels of the RTL gene, and the control plants.

### Example 2

### Maize and Arabidopsis ARGOS Proteins Interact with AtRTE1 in Arabidopsis

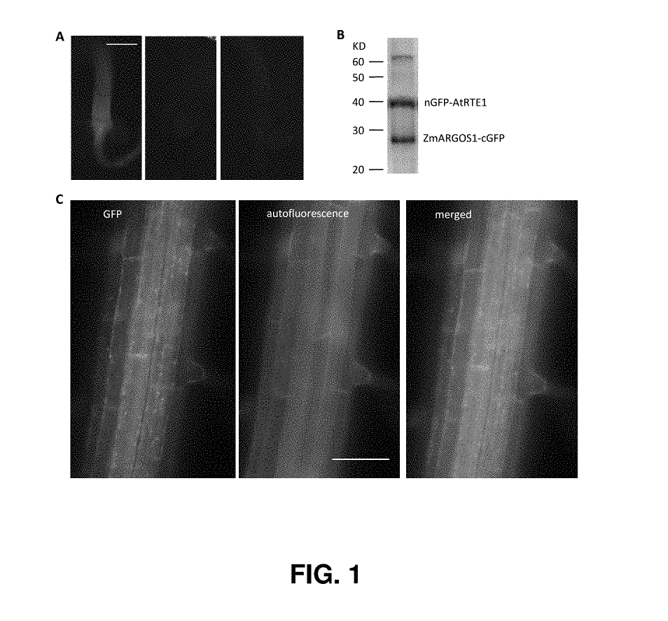

Genetic analysis indicated that ZmARGOS1 targets the ethylene signaling pathway between the ethylene receptors and CTR1 (Shi et al., 2015, supra). To test if ZmARGOS1 physically interacts with Arabidopsis RTE1, the sequence encoding for the N- and C-terminal halves of split Aequorea coerulescens green fluorescent protein (nGFP and cGFP) was fused in frame to AtRTE1 at the N-terminus and ZmARGOS1 at the C-terminus, respectively. The fusion genes were introduced individually into Arabidopsis to generate stably transformed events. The ER-localized membrane protein AtRTE1 has a cytosolic N-terminus. Both the N- and C-termini of ZmARGOS1 are predicted (PRODIV-TMHMM; Viklund and Elofsson, 2004) to expose to cytosol. Overexpression of the ZmARGOS1-cGFP transgene reduced ethylene sensitivity in Arabidopsis, and so did nGFP-AtRTE1, indicating that the split GFP-tagged proteins retain their function. The nGFP-AtRTE1 transgenic plants did not show green florescence, nor those overexpressing the ZmARGOS1-cGFP (FIG. 1A), as expected. However, when the two constructs were brought together by crossing the transgenic plants, both ZmARGOS1-cGFP and nGFP-AtRTE1 fusion proteins were detectable in F1 plants with Western blotting (FIG. 1B) and GFP-positive florescence signals was observed in the F1 etiolated seedlings (FIG. 1A), indicating protein-protein interactions between ZmARGOS1 and AtRTE1. Using standard bimolecular fluorescence complementation assay (BiFC), it was found that Arabidopsis ARGOS homolog ORGAN SIZE RELATED1 (OSR1) also interacts with AtRTE1. Fluorescence microscopy of the hypocotyl cells showed that the fluorescence signals were associated with interconnected threads and small bodies in the cytoplasm (FIG. 1C), consistent with the subcellular localization of the ARGOS and AtRTE1 proteins in the endoplasmic reticulum (ER) and Golgi (Shi et al., 2015, supra).

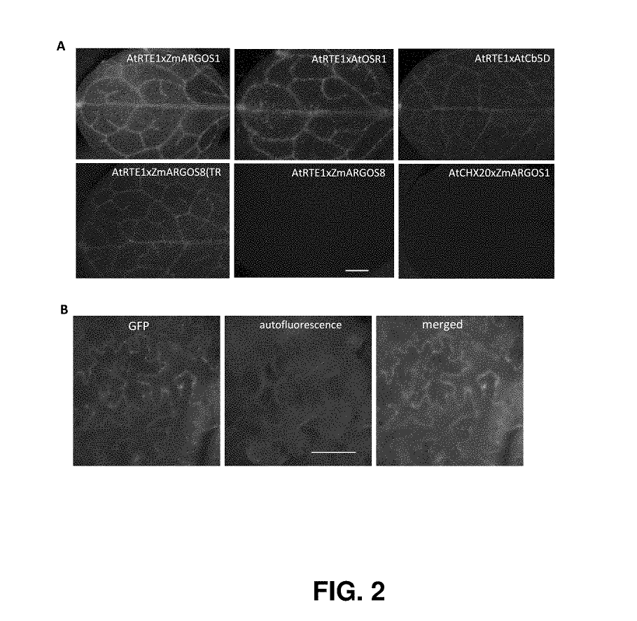

In leaves of F1 plants derived from crosses of the DMMV:ZmARGOS1-cGFP and DMMV:nGFP-AtRTE1 plants, reconstituted green fluorescence was observed in the epidermal cells (FIG. 2B), as well as in the vascular tissues which have strong signals (FIG. 2A). AtRTE1 was reported to interact with the ER-localized cytochrome b5 (AtCb5). Therefore, Arabidopsis Cb5 isoform D (AtCb5D) was used as positive control for the BiFC assay, and a similar pattern of fluorescence signals was found in the vascular tissues (FIG. 2A) and the epidermal cells. No BiFC signals was detected in F1 plants overexpressing ZmARGOS8-cGFP and nGFP-AtRTE1, but interactions between ZmARGOS8(TR) and AtRTE1 were evident, as reflected in the BiFC signals in the vascular tissues (FIG. 2A).

### Example 3

### Interaction of ARGOS with Arabidopsis RTE1 and Maize Homologs in the Yeast Model System

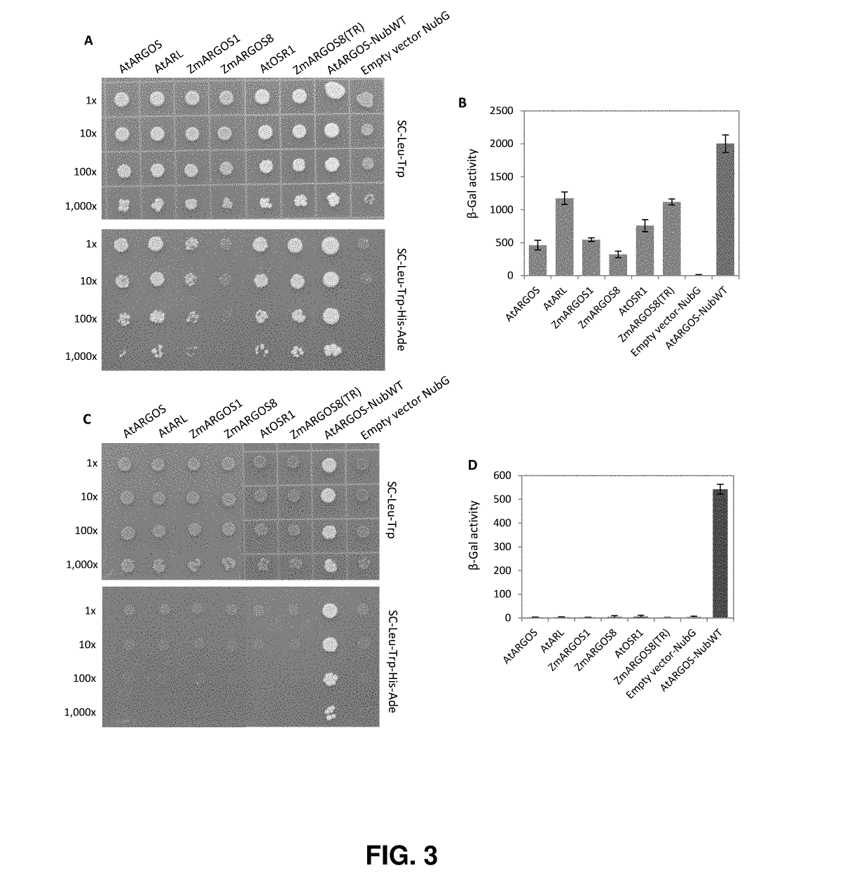

To confirm the protein-protein interactions of ZmARGOS1 and AtOSR1 with AtRTE1, a mating-based split-ubiquitin yeast two-hybrid system was employed. The coding sequences of ZmARGOS1 and AtOSR1 (prey) were fused in frame to the N-terminal half of a mutated ubiquitin (NubG; containing mutation Ile13Gly). AtRTE1 (bait) was cloned as a translational fusion to the C-terminal half of ubiquitin (Cub) followed by a synthetic transcription factor, PLV (protease A-LexA-VP16). The NubG with reduced affinity to the Cub moiety is unable to reconstitute functional ubiquitin. Only when the bait and prey proteins interact at the ER membranes, the NubG is brought into the vicinity of the Cub domain in the cytosol side of the ER membrane, forming a functional ubiquitin. Endogenous ubiquitin-specific proteases then release the transcription factor, which diffuses into the nucleus where it activates the transcription of reporter genes (HIS3, ADE2 and lacZ). Expression of the AtRTE1-Cub-PLV fusion protein, as well as cleavage and function of the LexA-VP16 transcription factor, was verified by pairing the bait construct with a prey construct containing the N-terminal domain of wild-type ubiquitin (NubWT) (FIGS. 3 A and B). The NubWT interacts with Cub independent of the prey-bait association, reconstituting ubiquitin and activating reporters. Empty NubG vector, which expresses a soluble NubG, serves as controls to eliminate the possibility of self-activation of the AtRTE1-Cub-PLV fusion protein (FIG. 3).

Yeast diploid cells produced by mating yeast strains containing the ZmARGOS1 and AtOSR1 prey constructs with the strain containing the AtRTE1 bait construct can grow on the synthetic complete (SC)-Leu-Trp-His-Ade medium (FIG. 3A), indicating protein-protein interactions between the bait and prey. Lacking of red pigment accumulation in the diploid cells grown on the SC-Leu-Trp medium indicates that the transcription of the reporter gene ADE2 was also activated (FIG. 3A), consistent with the results from the HIS3-dependent growth assay. Interactions of ZmARGOS1 and AtOSR1 with AtRTE1 were further verified with the β-galactosidase assay (FIG. 3B), which measures the activity of the LacZ reporter. As a negative control, Arabidopsis CHX20 was fused in frame to the Cub-PLV to produce a bait construct which was used in mating experiments with the ARGOS prey constructs (FIG. 3C). No apparent growth under histidine selective conditions were observed in these matings, nor was β-galactosidase activity detected (FIG. 3D). The diploid cells grown on the SC-Leu-Trp medium accumulated a red pigment (FIG. 3C), indicating that the reporter gene ADE2 was inactive as well. Taken together, the data suggested that ZmARGOS1 and AtOSR1 physically interact with the ethylene receptor regulator AtRTE1 in yeast, corroborating the BiFC results obtained in Arabidopsis.

Using the same yeast system, we found that Arabidopsis ARGOS and ARGOS-LIKE (ARL) also interact with AtRTE1 (FIG. 3). Yeast diploid cells expressing ZmARGOS8-NubG grew less than the cells expressing other ARGOS-NubG fusion proteins in the interaction-dependent growth assay (FIG. 3A) and concomitantly showed lower β-galactosidase activity (FIG. 3B), indicating a weak interaction between ZmARGOS8 and AtRTE1. The truncation variant ZmARGOS8(TR), however, displayed a strong interaction with AtRTE1 (FIGS. 3A and B), consistent with the results of the BiFC assay in Arabidopsis and its high activity in conferring ethylene insensitivity in Arabidopsis and maize plants. With the establishment of interactions between AtRTE1 and various ARGOS, we next tested maize RTE1 homologs.

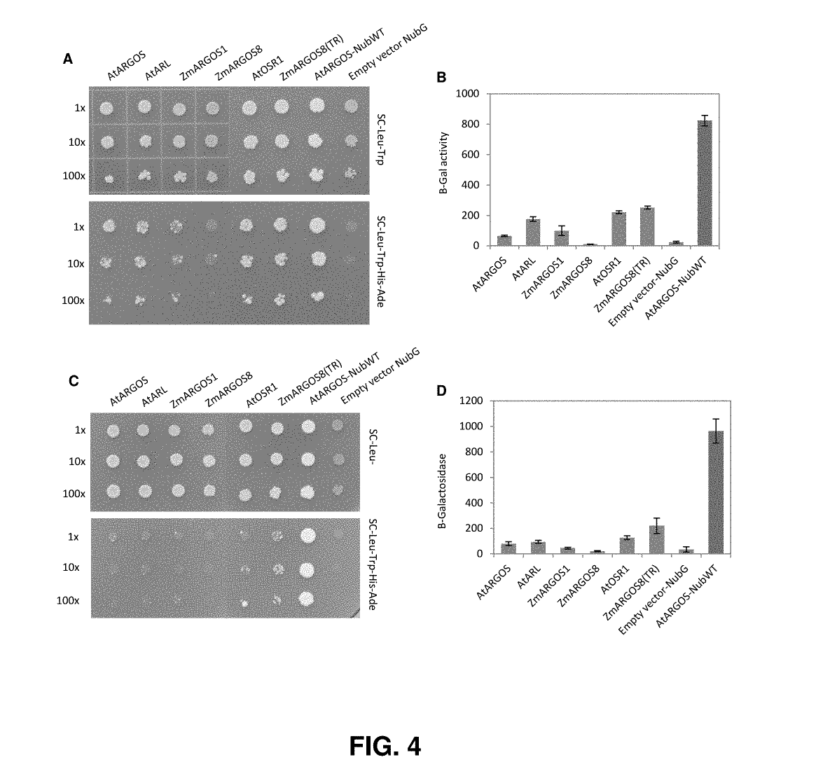

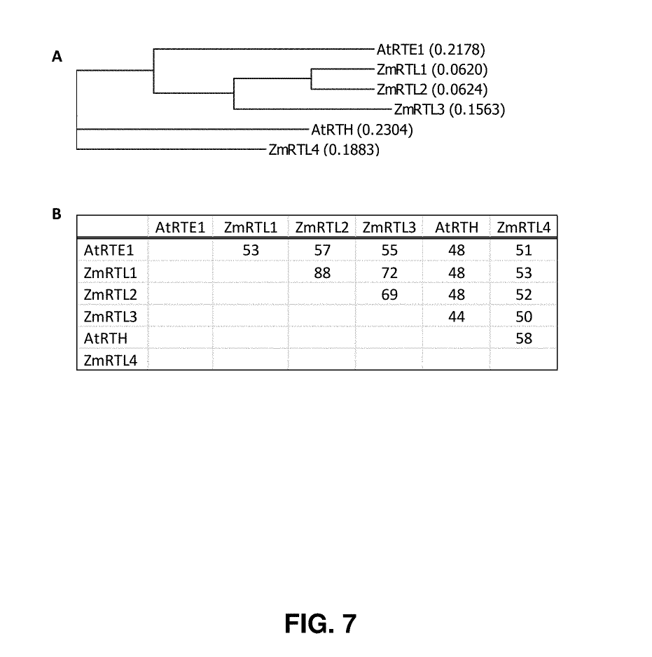

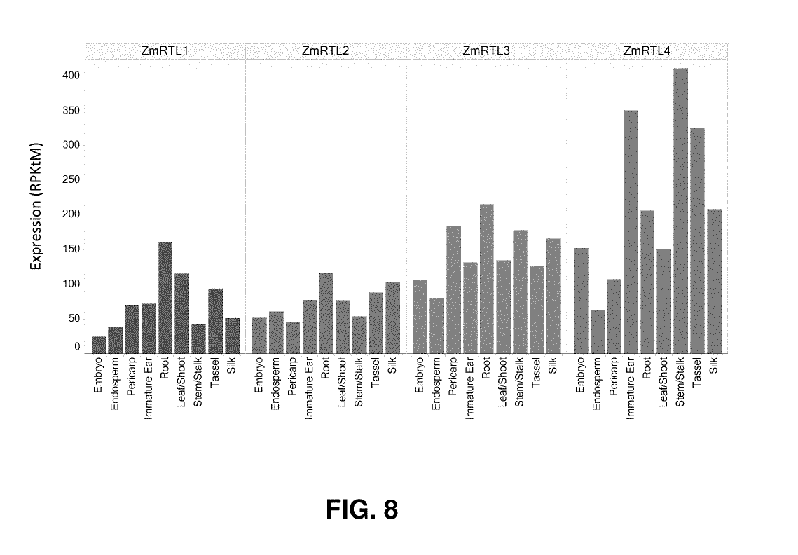

BLAST search with AtRTE1 protein sequence revealed four homologous genes in maize genome (FIG. 7). We designated these genes as REVERSION-TO-ETHYLENE SENSITIVITY1 LIKE1 (RTL1), RTL2, RTL3 and RTL4. Their amino acid sequences are 52%, 53%, 50% and 42% identical to AtRTE1, respectively, in pairwise comparison. They express broadly across various tissues in maize (FIG. 8) and their function are unknown. ZmRTL2 and ZmRTL4 were cloned into the bait construct, paired with the ARGOS preys, and tested for protein-protein interactions in the yeast split-ubiquitin two-hybrid system. Data presented in FIG. 7 revealed that ZmRTL4 interacts with ZmARGOS1, ZmARGOS8(TR) and three Arabidopsis ARGOS proteins. Protein-protein interactions between ZmARGOS8 and ZmRTL4 apparently are weak, as indicated in growth of diploid cells on SC-Leu-Trp-His-Ade selective medium (FIG. 4A). With the same assay, ZmRTL2 was found to interact with ZmARGOS8(TR) and AtOSR1 (FIGS. 4, C and D). A weak interaction with AtARGOS, AtARL and ZmARGOS1 also is evident (FIGS. 4, C and D).

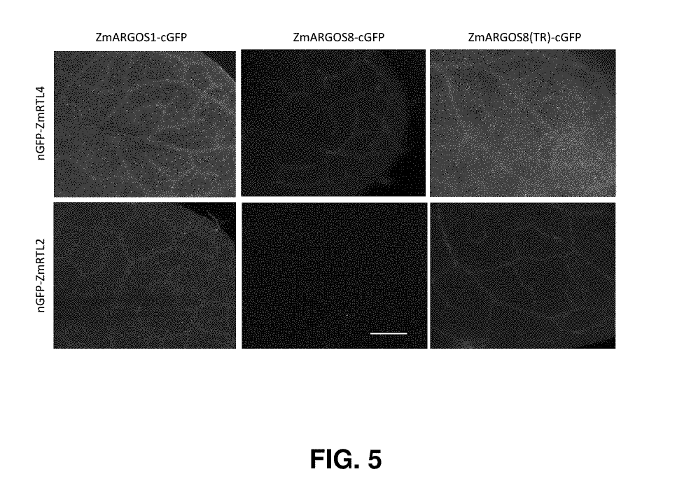

To verify the protein-protein interaction of maize ARGOS and RTL, BiFC was performed using stably transformed lines of Arabidopsis as described above. Reconstituted green fluorescence was observed in leaves of F1 plants derived from crosses of the DMMV::ZmARGOS1-cGFP and DMMV::nGFP-ZmRTL4 plants (FIG. 5). When nGFP-ZmRTL4 was brought together with cGFP-tagged ZmARGOS8(TR) or ZmARGOS8, BiFC signals were detected in both, but the signal from the ZmARGOS8(TR) combination was stronger than the full-length ZmARGOS8 (FIG. 5). ZmRTL2 also interacts with ZmARGOS1 and ZmARGOS8(TR), but no BiFC signals were detected in F1 plants of the ZmARGOS8 and ZmRTL2 crosses (FIG. 5).

### Example 4

### Over-Expression of Maize RTL Genes Reduces Ethylene Sensitivity in Maize

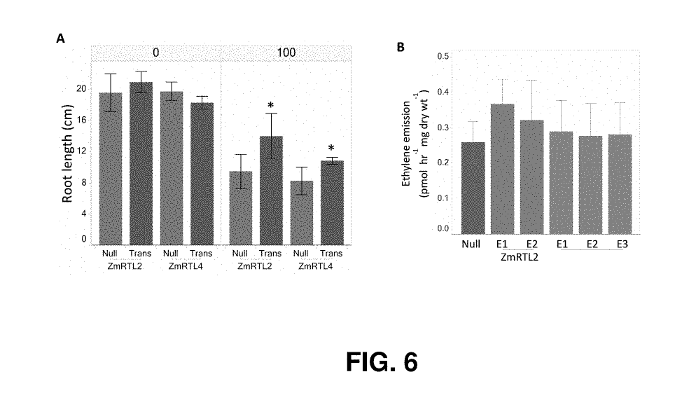

Arabidopsis RTE1 confers ethylene insensitivity when overexpressed in Arabidopsis. To determine the effect of maize AtRTE1 homologs on ethylene response, ZmRTL2 and ZmRTL4 were overexpressed in transgenic maize plants under the control of maize UBI1 promoter. Multiple single-copy events were generated for each construct in an inbred line, ZmRTL transgene expression confirmed by RT-PCR, and hybrid seeds produced by topcrossing the transformants to a tester inbred. The ethylene responsiveness of the transgenic plants was assessed by measuring primary root lengths in etiolated seedlings in the presence of 100 μM ACC. Data presented in FIG. 6A shows that overexpression of ZmRTL2 and ZmRTL4 alleviates the inhibitory effect of ACC on root growth, indicating reduced ethylene sensitivity in the transgenic plants.

The effect of overexpressing ZmRTL2 and ZmRTL4 on ethylene biosynthesis was determined in maize. No difference was found in ethylene emission rate between the ZmRTL transgenic plants and non-transgenic controls (FIG. 6B).

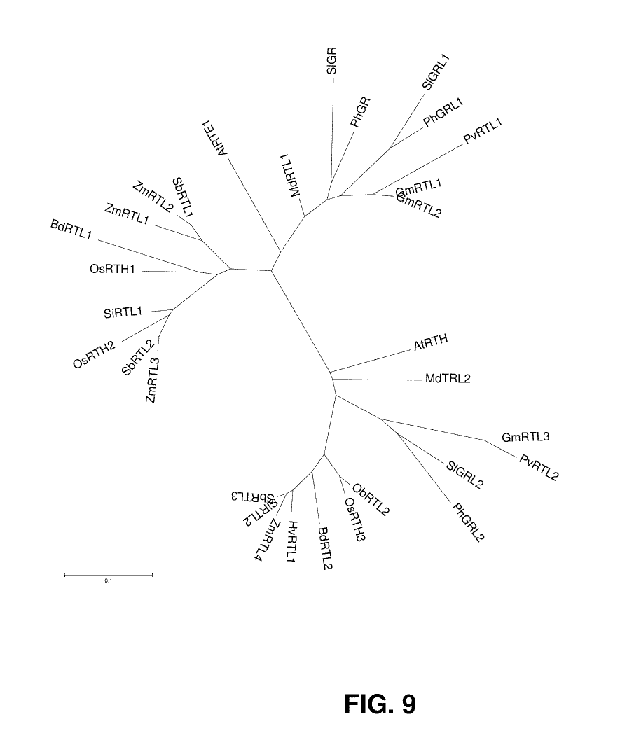

ZmRTL4 is more closely related to the AtRTH/SIGRL2 clade while ZmRTL2 belongs to the AtRTE1/SIGR clade (FIG. 9). However, both ZmRTL2 and ZmRTL4 can reduce ethylene sensitivity when overexpressed in maize. In addition, they were not able to complement Arabidopsis rte1-2 mutant when expressed under the control of the cauliflower mosaic virus 35S promoter (35S) or to reduce ethylene sensitivity in Arabidopsis, as measured in ethylene triple response assay and a root growth assay using light-grown seedlings. The finding of the UBI1:ZmRTL4 maize having reduced ethylene sensitivity suggests that the function of regulating ethylene signaling is not limited to the members of the AtRTE1/SIGR clade. Even though evolution of plant RTE1 gene family may have taken two separate routes, members from both clades apparently acquire the function to regulate the same signaling pathway. The evolutionary acquisition of the function may be caused by changes either in the RTE1 homologs themselves or in their interacting partners, such as the ethylene receptors, ARGOS proteins and Cb5s.

The N- and C-terminal regions are not conserved among the ARGOS family members, and they are not required for the activity of conferring ethylene insensitivity and the binding of AtRTE1 or ZmRTL4, as revealed by the truncation variant ZmARGOS1(TR-nc) and ZmARGOS8(TR). However, the two transmembrane helices (TM1 and TM2) and the Pro-rich motif (PRM) are necessary for the ARGOS activity. The TM1-PRM-TM2 (TPT) domain alone is sufficient to confer ethylene insensitivity in Arabidopsis and maize when overexpressed, and can bind to AtRTE1 and ZmRTL4, as shown in the BiFC assay and the yeast split-ubiquitin two-hybrid assay. Given the membrane localization of the protein-protein interaction, it is possible that the two transmembrane helices of ARGOS proteins are responsible for the association with AtRTE1 which is predicted to contain two or four transmembrane domains. In this scenario, the PRM may function as a linker to properly position the two transmembrane helices, forging a functional conformation for ARGOS. Substitution of Leu or Pro in this region would disturb the relative position of the two transmembrane helices, inactivating ARGOS, as observed in the mutation analysis. Alternatively, the PRM, predicted to be exposed to the luminal side of the ER, may function as a determinant for ARGOS in the protein-protein interactions. The Pro-rich regions in proteins preferentially adopt a polyproline type II helical conformation that facilitates transient intermolecular interactions.

Reduced ethylene signaling may be involved in the enhanced cell elongation and/or division in the ARGOS transgenic plants. Drought stress often slows down plant growth and even affects development, leading to grain yield loss in crops. These changes at whole plant levels are largely due to reduced cell expansion and/or division. Overexpressed ARGOS likely counteracts the effect of water deficiency by promoting cell expansion and/or division via modulating ethylene signal transduction, mitigating the yield loss by supporting plant growth under drought stress.

### Example 5

### Field Testing of ZmRTL Transgenic Plants for Yield Performance

Six single-copy events per UBI1ZM::ZmRTL construct were tested in a hybrid background at multiple locations. At the end of the growing season, locations were categorized into well-watered as well as low, medium and severe drought stress environments based on several drought stress parameters (Loffler et al., 2005, Crop Sci 45: 1708-1716). Grain yield was analyzed using a mixed model via known ASRemI. The transgenic plants had the same yield as the null controls (Table 2).

TABLE 2
Field testing of ZmRTL transgenic plants for yield performance

| | | Well- | Low | Medium | Severe |
|---|---|---|---|---|---|
| | | watered | Stress | Stress | Stress |
| Construct | Genotype | bu/ac | | | |
| Bulked Null | Non-transgenic | | 167.32 | 139.61 | 81.26 |
| ZmRTL1 | Transgenic | | 166.44 | 141.84 | 82.48 |
| ZmRTL2 | Transgenic | | 166.88 | 136.44 | 80.92 |
| ZmRTL3 | Transgenic | | 164.79 | 142.33 | 83.54 |
| Bulked Null | Non- | 234.44 | 176.46 | 141.22 | 83.91 |
| | transgenic | | | | |
| ZmRTL4 | Transgenic | 234.83 | 177.33 | 139.56 | 77.01* |

*t-Test, p < 0.1

What is claimed is:

1. A maize plant in which expression of a RTL gene or activity of a RTL polypeptide is increased, when compared to a control plant, wherein the RTL gene encodes the RTL polypeptide that comprises an amino acid sequence with at least 90% sequence identity to SEQ ID NOS: 1-4 and wherein the plant exhibits at least one phenotype selected from the group consisting of: reduced ethylene sensitivity, increased yield, increased drought stress tolerance, and increased biomass compared to the control plant not expressing the RTL gene.

2. The plant of claim 1, wherein the RTL gene is an endogenous gene encoding a polypeptide sequence comprising an amino acid sequence that is at least 95% identical to SEQ ID NO: 1, and the increase in expression is caused by insertion of a heterologous regulatory element.

3. The plant of claim 1, wherein the RTL gene is an endogenous gene encoding a polypeptide sequence comprising an amino acid sequence that is at least 95% identical to SEQ ID NO: 1, and the increase in expression is caused by a mutation in the endogenous regulatory element that increases the expression of the endogenous RTL gene.

4. The plant of claim 2, wherein the endogenous RTL gene is altered by a zinc finger nuclease, Transcription Activator-Like Effector Nuclease (TALEN), guided Cas9 endonuclease or meganuclease.

5. The plant of claim 1, wherein the RTL polypeptide activity is increased as a result of mutation of an endogenous RTL gene.

6. A DNA construct comprising a polynucleotide, wherein the polynucleotide encodes a polypeptide comprising a sequence that is at least 95% identical to SEQ ID NO: 1, is operably linked in sense orientation to a heterologous promoter, wherein the expression of the polynucleotide in a maize results in reduced ethylene sensitivity as compared to a control plant not expressing the polypeptide.

7. The DNA construct of claim 6, wherein the heterologous promoter is maize GOS2 promoter.

8. A method of making a plant in which expression of an endogenous RTL gene is increased, when compared to a control plant, and wherein the plant exhibits at least one phenotype selected from the group consisting of: reduced ethylene sensitivity, increased yield, increased abiotic stress tolerance, and increased biomass, compared to the control plant, the method comprising the steps of introducing into a plant a DNA construct comprising a polynucleotide operably linked to a heterologous promoter, wherein the DNA construct is effective for increasing expression of an endogenous RTL gene.

9. The method of claim 8, wherein the DNA construct comprises a heterologous regulatory element.

10. The method of claim 9, wherein the heterologous regulatory element is a promoter.

11. The method of claim 9, wherein the heterologous regulatory element is maize GOS2 promoter.

12. A method of identifying one or more alleles associated with increased yield in a population of maize plants, the method comprising the steps of:
(a) detecting in a population of maize plants one or more polymorphisms in (i) a genomic region encoding a polypeptide or (ii) a regulatory region controlling expression of the polypeptide, wherein the polypeptide comprises the amino acid sequence selected from the group consisting of SEQ ID NOS: 1-4, or a sequence that is 90% identical to SEQ ID NOS: 1-4, wherein the one or more polymorphisms in the genomic region encoding the polypeptide or in the regulatory region controlling expression of the polypeptide is associated with yield; and
(b) identifying one or more alleles at the one or more polymorphisms that are associated with increased yield.

13. The method of claim 12, wherein the one or more alleles associated with increased yield is used for marker assisted selection of a maize plant with increased yield.

14. The method of claim 12, wherein the one or more polymorphisms is in the coding region of the polynucleotide.

15. The method of claim 12, wherein the regulatory region is a promoter.


SEQUENCE LISTING (12 sequences)

SEQ ID NO: 1 - protein, 204 aa, Zea mays
MENDRRQLGQIDPRRARFPCCIVWTPIPFITWLVPFIGHIGICREDGVILDFSGPHFVSVDNFAFGAVARYIQVNCDECYKLIEPEGDATWDGALKKGTQEFQNRNYNLFTCNCHSFVANNLNRLFYSGHDEWNVVSLAAVMFLRGRWVSTASVVKTLLPFAVVLSIGTFLGGTTFLTGLLAFAAAMTVWFLVGTYCIKGLIEL

SEQ ID NO: 2 - protein, 236 aa, Zea mays
MSPKVLSSMEVEAGFAGDGVGSNNGPQDMWSLGEIDPKRARFPCCIVWTPLPVVSWLAPYIGHVGICQEDGAVLDFAGSNLVSVDNFAYGSVARYLQLDRKKCCLPVNLAEHVCKQSYNHSELGAAISWDDALRSSMRRFQHKYYNLFTCNCHSFVASCLNRLAYNGSLEWNVLNVAALVWFHGRWVDRMSSVRSFLPVLPVTCIGILMAGWSFLLGMAAFSALSDWMVCFHSVLR

SEQ ID NO: 3 - protein, 233 aa, Zea mays
MELEADFADEDVSSNNGLQDLWSLDEIDSKRARFPCCIVWTPLPVVSWLAPYIGHVGICQEDGAILDFAGSNLVSMDNFAYGSVARYLQLDRKKCCLPVNLAAHVCKQSYSHSEVGAAMSWDDALQSGMRRFQHKYYNLFTCNCHSFVANCLNRLAYNGSVEWNVLNVASLVWFHGQWVDKMSFARSFLPFLTVTCIGILMAGWSFLVGMAAFSILLIGWFVFTVYCVKGLIC

SEQ ID NO: 4 - protein, 233 aa, Zea mays
MELEAAPDDKVFCSDDEMQTLWPLGQVDPKSVRFPCCIVWTPLPVVSWLAPYIGHVGIAREDGTVLDFAGSNLVSVDDLAYGSVARCLQLDRAKCCFPANPASHVCLRSHEHSDAGTAISWDDALRSGSRRFEHKCYNLFTCNSHSFVADCLNRLAYGGSVGWNVLNLAALVWLRGRWLDPMAAVRSFLPFAVVSCVGVLMAGWSFLLGMTAFTLLLLGWFVLGVYCMKGLVV

SEQ ID NO: 5 - DNA, 615 bp, Zea mays
atggaaaatgatagaaggcaacttggtcagatcgatccaagaagagcccgctttccttgctgcatagtatggactccaataccattcatcacttggttggtgccctttatcggtcacattggcatctgcagagaagacggtgtaattctggacttctctggtccacattttgtatcagttgacaattttgcattcggagctgttgcgcgctacattcaagtaaactgcgatgagtgctataagcttattgaacctgaaggagatgccacgtgggacggcgcgctgaagaaaggcacgcaggagttccaaaacaggaactacaacctgttcacctgcaactgccactcctttgtcgcaaacaatctgaacaggctgttttattccggccacgacgaatggaatgtggtcagcttggctgctgtgatgttcttacgaggccgctgggtgagcacggcatcggtggtgaagaccttattgccatttgcagttgtgctttccatcggtaccttccttggcggcacgaccttcctgaccggccttcttgctttcgccgctgcgatgactgtctggttccttgtgggcacctactgcatcaaaggtctcatagagttgtga

SEQ ID NO: 6 - DNA, 711 bp, Zea mays
atgtcacctaaagtactttcctcgatggaggttgaagctggttttgctggcgatggcgtcggttcaaataatggaccacaagacatgtggtcacttggcgagatagatccaaaaagagcaaggttcccgtgctgcattgtctggactcctcttcctgtagtttcatggcttgctccttacatagggcatgtcggaatctgtcaggaggacggggctgtcttggattttgctggttcgaatttggtgagcgtggataattttgcttatggttcagttgccagatacctccagcttgacagaaagaagtgctgccttcccgttaatcttgcagaacacgtatgcaagcagtcttacaaccattcagaattaggagcagcgatatcatgggacgatgctctgcgatcgagcatgagacgcttccagcacaagtactacaacctgttcacctgcaactgccactcgtttgtggcgagctgcctgaaccggcttgcctacaatggctccctggagtggaatgtcttgaacgtggctgcccttgtctggtttcatggccgatgggtggacagaatgtcttccgttcggtcgttcttacctgtcctacccgtgacatgcatcggcatcttaatggctggctggtctttcctgctagggatggcagcgttctctgctctttctgattggatggtttgttttcacagtgtactgcgttaa

SEQ ID NO: 7 - DNA, 702 bp, Zea mays
atggagcttgaagctgatttcgctgatgaagatgtcagttcaaataatggattacaagatctgtggtcacttgacgagatagattcaaagagagcaagattcccgtgctgcattgtttggactcctcttcctgtagtttcatggcttgccccttacatagggcatgttggaatctgtcaggaggatggggctatcttggattttgctggttcaaatttggtgagcatggataattttgcttatggttcagttgccagatacctccagcttgacagaaagaagtgctgccttcctgttaatcttgcagcacatgtatgcaagcagtcctacagccattcagaagttggagcagcgatgtcatgggatgatgctctgcaatcgggcatgagacgcttccagcacaagtactacaatctgttcacctgcaattgccactcgtttgtggcaaactgcctgaaccggctcgcttacaatggctctgtggagtggaatgttttgaatgtggcttcccttgtttggtttcatggccaatgggtggacaaaatgtctttcgctcggtctttcttgcctttcctaactgtgacatgcatcggtattttaatggctggctggtctttcctggtagggatggcagcattctctattcttttgattggatggtttgtttttacggtatactgcgttaagggtttgatatgttga

SEQ ID NO: 8 - DNA, 702 bp, Zea mays
atggagcttgaagctgctcctgacgataaagtattctgttcagatgatgagatgcagacgctgtggcctctgggacaagtagacccaaagagcgtgaggttcccttgctgcatcgtgtggactcctctcccagtagtttcatggctggctccctacatagggcacgtggggattgctcgggaggatggaaccgtcttggactttgcaggttcgaatttagtgagtgtggatgatctggcttatggttctgtcgccagatgcctgcagcttgacagggcaaagtgctgcttccctgccaacccggcgtcgcacgtgtgcttgaggtcccacgagcactcggatgccgggacggcgatctcgtgggacgacgcgctgcggtcggggagccggcgcttcgagcacaagtgctacaacctcttcacctgcaacagccactcgttcgtggccgactgcctcaaccggctggcttacggcggctccgttgggtggaacgtgctgaacctggccgcgctcgtctggctgcgcggccggtggctggaccccatggccgccgtccggtccttcctcccgttcgccgtcgtctcctgcgtcggcgtcctgatggccggctggtccttcctcctgggcatgaccgcgttcacgctgctcttgctcggctggttcgtgctcggcgtctactgcatgaagggcctcgtcgtctga

SEQ ID NO: 9 - DNA, 1255 bp, Zea mays
accgtttgggtcgctagacggtcgccacgcccgcggtccagcgttggcatccgtcctgcaagcctgcaacccaaccttgaaccttgcctgttcttcttcgatttattagcccagcgttctccctctctcgtgggcgtggcgacgacttggaggccatcgctgccgccctacgccaggtgcccagatcatctgcggccccttcgccggcgacgatcaccatccatcaggtacgtggtacgcccagcccttgctcttcccttcctgctgtgagaaacagggcatccaaaccctatcttgctatttgcatccggatcttatctaaatagcgtatatatgcatgtattatatgcctactaacttggattctttcccaaaattatcatttccattactgggtccgcccctggttggatcgctggttttggggcaacgcctcgtccataaataggatcaagcgcttgaacttacagcgagatggaaaatgatagaaggcaacttggtcagatcgatccaagaagagcccgctttccttgctgcatagtatggactccaataccattcatcacttggttggtgccctttatcggtcacattggcatctgcagagaagacggtgtaattctggacttctctggtccacattttgtatcagttgacaattttgcattcggagctgttgcgcgctacattcaagtaaactgcgatgagtgctataagcttattgaacctgaaggagatgccacgtgggacggcgcgctgaagaaaggcacgcaggagttccaaaacaggaactacaacctgttcacctgcaactgccactcctttgtcgcaaacaatctgaacaggctgttttattccggccacgacgaatggaatgtggtcagcttggctgctgtgatgttcttacgaggccgctgggtgagcacggcatcggtggtgaagaccttattgccatttgcagttgtgctttccatcggtaccttccttggcggcacgaccttcctgaccggccttcttgctttcgccgctgcgatgactgtctggttccttgtgggcacctactgcatcaaaggtctcatagagttgtgattagagtatgttagactcgttgtagggggttcaagggtggcatatttttaagagttcaagcgtggcacatttttatttgtattgcattcctgtataatggattcttgatgttattcatgcacctatatgttccaaattccgaatgaattgatgcacttgtctgttg

SEQ ID NO: 10 - DNA, 1205 bp, Zea mays
tgcttgcactcttacgtccacgttcggtgtattacatagtgacattgccatatgaaattgtaaagtaaataaaggttgataagctgtgaggttgaagctagagacgccgctgttttaagtttcttcaaggacaagggaacattaatgtcacctaaagtactttcctcgatggaggttgaagctggttttgctggcgatggcgtcggttcaaataatggaccacaagacatgtggtcacttggcgagatagatccaaaaagagcaaggttcccgtgctgcattgtctggactcctcttcctgtagtttcatggcttgctccttacatagggcatgtcggaatctgtcaggaggacggggctgtcttggattttgctggttcgaatttggtgagcgtggataattttgcttatggttcagttgccagatacctccagcttgacagaaagaagtgctgccttcccgttaatcttgcagaacacgtatgcaagcagtcttacaaccattcagaattaggagcagcgatatcatgggacgatgctctgcgatcgagcatgagacgcttccagcacaagtactacaacctgttcacctgcaactgccactcgtttgtggcgagctgcctgaaccggcttgcctacaatggctccctggagtggaatgtcttgaacgtggctgcccttgtctggtttcatggccgatgggtggacagaatgtcttccgttcggtcgttcttacctgtcctacccgtgacatgcatcggcatcttaatggctggctggtctttcctgctagggatggcagcgttctctgctctttctgattggatggtttgttttcacagtgtactgcgttaagggtttggtatgttgactggaaaaagaattactgaaagcactccattagtgcattgcagtctaggaattatggatgtaatcgctgtgctggagtcaggtaaatcactcgtgtcctcagcctcagtgattcagacctgtgcaagttcgtcaaacaaaaaaattgacctatggaggtggtctggagataatttggttcatggcaagttcgtcaatttatttagtgctgttccaaggaaaaaagaaattcttttgattcaagcatctctttctgaacttaacaacttgatgtattagttttatatttaatattccaagaaatcacatcatagtatatcaatgctctctaatat

SEQ ID NO: 11 - DNA, 1443 bp, Zea mays
ctgataagaacgtaatgatagctttccaatttgtgcatgggtgcctcctcagatatggcaaaattaacgcctggtccatctgtgcccaaagtacaaatggtgacatgctaatctttttctggtggcgtgtgttggccgttgggagaaagatttgtattagtggtaaagatattcacatggttcaggacccattgcttgtacttttatgtccacattcaatgtattgcatagtgatgttgccgtatgaaattgtaaaggtagtgtactaaactaggtgcacatttttaatccttcaccagttgttttaatcatgaagattgagaacctaaccggatagctccttttcagtgaaggtcgataaactgtgagatactgagattgaagctgcacactgttttagtttcttcaaggacaaggaacattaatatcaccaaatactttcgtcaatggagcttgaagctgatttcgctgatgaagatgtcagttcaaataatggattacaagatctgtggtcacttgacgagatagattcaaagagagcaagattcccgtgctgcattgtttggactcctcttcctgtagtttcatggcttgccccttacatagggcatgttggaatctgtcaggaggatggggctatcttggattttgctggttcaaatttggtgagcatggataattttgcttatggttcagttgccagatacctccagcttgacagaaagaagtgctgccttcctgttaatcttgcagcacatgtatgcaagcagtcctacagccattcagaagttggagcagcgatgtcatgggatgatgctctgcaatcgggcatgagacgcttccagcacaagtactacaatctgttcacctgcaattgccactcgtttgtggcaaactgcctgaaccggctcgcttacaatggctctgtggagtggaatgttttgaatgtggcttcccttgtttggtttcatggccaatgggtggacaaaatgtctttcgctcggtctttcttgcctttcctaactgtgacatgcatcggtattttaatggctggctggtctttcctggtagggatggcagcattctctattcttttgattggatggtttgtttttacggtatactgcgttaagggtttgatatgttgaccggagaagaattactgaaagcactcattagcgcatcgcagtctacatgtaatcgctgtgctagattcaattaaataattgatgtcatcagcttcagtgattcagaaacctgttcaaattcagaccaatttattttgacttatggaggcggtctcgtgaaaaatttagttcatggcgtgctcattggcctcacaatcttgtttctgcaattgcatggcctaacaacaaaggtacgcactctcctgcacattttcgagatagaaaaaaattgtcacaatctttgctcattgtagta

SEQ ID NO: 12 - DNA, 1529 bp, Zea mays
ctcctccctcccctcccctcccctcccctcccggctcccgcacgctataaactgtagctgccgcggcgcctgcttctccccgatgcgtcaaacacttgacctgcacacggacagcggcggtggcgtcccgatcctgtccacccacccatctaatgcggccgcgaggaggatccacctgattctgctgctcctgctcctccttctagtcgagagaccatgacacggaagtcagcagtagcggattatgtttagtggcacaaggatcattcgtgtagctgaaaggatttgccttcaatggagcttgaagctgctcctgacgataaagtattctgttcagatgatgagatgcagacgctgtggcctctgggacaagtagacccaaagagcgtgaggttcccttgctgcatcgtgtggactcctctcccagtagtttcatggctggctccctacatagggcacgtggggattgctcgggaggatggaaccgtcttggactttgcaggttcgaatttagtgagtgtggatgatctggcttatggttctgtcgccagatgcctgcagcttgacagggcaaagtgctgcttccctgccaacccggcgtcgcacgtgtgcttgaggtcccacgagcactcggatgccgggacggcgatctcgtgggacgacgcgctgcggtcggggagccggcgcttcgagcacaagtgctacaacctcttcacctgcaacagccactcgttcgtggccgactgcctcaaccggctggcttacggcggctccgttgggtggaacgtgctgaacctggccgcgctcgtctggctgcgcggccggtggctggaccccatggccgccgtccggtccttcctcccgttcgccgtcgtctcctgcgtcggcgtcctgatggccggctggtccttcctcctgggcatgaccgcgttcacgctgctcttgctcggctggttcgtgctcggcgtctactgcatgaagggcctcgtcgtctgacgcacgctggctcggagccgaacgtgtgcccagcttcttcactgagcatcggggtcatgtagtagatgaatgaaggatcgaaatcttgaaaggggtgtgagtcggaggggagagacaacgggaaaacaaaatttgagcccgtttttaatagcggaaacccaaatccagatatttataaaatcgaagtttctatttgaaacaaagtagaagtacaagataaaaggaaggagggggaaatagcacaaaaacaaaattgggctagagtcttagaaccgttggaggccgaaactcacgccctccttgctgcgaagctgtcgatctgcagaatgcagctctgttaactgacagtctcctgttacgaggcaatcagaggctttttctagactggacctcgccatatccaccatatagatataggaggtaccagaagggggtgttctatatagcaagctgtagctgaactcattggattagacatatcagggcgatcaatccaatagcatctcagtgtcctgtcatttcataaaaaaaca